(19) United States
(12) Patent Application Publication
(10) Pub. No.: US 2003/0190335 A1
(43) Pub. Date: Oct. 9, 2003

(54) COSMETIC USE OF SELECTED POLYAMINO POLYMERS AS ANTIOXIDANTS

(76) Inventors: BOUDIAF BOUSSOUIRA, PARIS (FR); CHRISTIAN COLIN, PARIS (FR)

Correspondence Address: FINNEGAN HENDERSON FARABOW, GARRETT & DUNNER, 1300 I STREET N W, WASHINGTON, DC, 200053315

(21) Appl. No.: 09/083,150

(22) Filed: May 22, 1998

(30) Foreign Application Priority Data
May 22, 1997 (FR) 97 06258

(51) Int. Cl. A61K 31/74; A61K 31/785; A61K 31/795; A61K 6/00; A61K 7/00

(52) U.S. Cl. 424/401; 424/78.18; 424/78.22; 424/78.35; 514/844; 514/887; 514/938; 514/939

(57) ABSTRACT

Use of a polyamino polymer to inhibit the light-induced peroxidation of lipids, particularly inhibition of the photo-peroxidation of lipids induced by nanopigments. The invention also relates to the use of a polyamino polymer to inhibit the light-induced peroxidation of proteins. This property of polyamino polymers finds applications in particular in the cosmetics, pharmaceutical, veterinary and agrifood fields.

Applicants reference herein the patent applications of BOUDIAF BOUSSOUIRA and DIDIER CANDAU for COMPOSITION COMPRISING A DIBENZOYLMETHANE DERIVATIVE AND A POLYAMINO POLYMER(Docket No. 05725.0304), and of BOUDIAF BOUSSOUIRA and DIDIER CANDAU for COMPOSITION COMPRISING A CINNAMIC ACID DERIVATIVE AND A POLYAMINO POLYMER (Docket No. 05725.0305), filed on even date herewith and incorporate the disclosures thereof specifically by reference herein.

The present invention relates to the use of a polyamino polymer as an antioxidant. The invention relates in particular to the use of a polyamino polymer in order to inhibit the light-induced peroxidation of lipids. This invention relates in particular to inhibition of the light-induced peroxidation of lipids of sebaceous origin (sebum), such as squalene, as well as to the light-induced peroxidation (or photo-peroxidation) of plant oils. The invention relates particularly to inhibition of the photo-peroxidation of lipids induced by nanopigments. The invention also relates to the use of a polyamino polymer in order to inhibit the light-induced peroxidation of proteins. This invention relates in particular to inhibition of the light-induced peroxidation of proteins of the skin, such as collagen, as well as to inhibition of the light-induced peroxidation (or photo-peroxidation) of proteins of animal or plant origin and derivatives thereof. This property of the polyamino polymers finds applications in particular in the cosmetic, pharmaceutical, veterinary and agrifood fields.

It is known that lipids found at the surface of the skin, the scalp and the hair are permanently subjected to external attack and in particular to air, atmospheric pollutants, visible radiation and especially ultraviolet (UV) radiation.

These lipids are both those which form part of the constituents of the skin or of the hair and those which are secreted by the skin, including the scalp, and/or those which are deposited on the skin or the hair during the application onto the latter of products containing lipids.

The lipids most exposed to external attack are those contained in the greasy secretions of the skin such as sebum, which is rich in squalene. The presence of six double bonds in squalene molecules makes these molecules sensitive to oxidation phenomena. Thus, during prolonged exposure to UV, squalene photo-peroxidizes to give squalene peroxides.

This high production of squalene peroxides results in particular in a series of chain degradations, in particular in and on the skin, giving rise to many skin disorders. Thus, these squalene peroxides are involved in particular in:

the pathogenesis of acne, as described by Saint Léger et al. (see British Journal of Dermatology, 1986: 114, pp. 535-542), who point out that squalene peroxides are comedogenic;

premature ageing of the skin as described by Keiko O. H. Sawa et al. (see The Journal of Toxicology Sciences, 1984: 19, pp. 151-159) who discuss the consequences of skin bums caused by the sun;

irritation phenomena as reported by Takayoshi Tanaka et al. (see J. Clin. Biochem. Nutr., 1986: 1, pp. 201-207), who report the damage caused in particular by the repeated use of certain shampoos;

the production of malodorous volatile products (aldehydes, ketones, acids, etc.) and, lastly, in

immunosuppression, as biochemical messengers of the biological effects of UV irradiation of the skin, as described by M. Picardo et al. (see Photodermatol. Photoimmunol. Photomed., 1991: 8, pp. 105-110).

It is known that the proteins found at the surface of the skin, the scalp and the hair are also subjected to external attack and in particular to the air, atmospheric pollutants and visible radiation and especially ultraviolet (UV) radiation. In this respect, mention may be made of the following documents: M. -L. Hu and A. L. Tappel, Photochem. Photobiol. 56 (3), 357-363, 1992; M. J. Davies et al., Biochem. J., 305, 643-649, 1995.

These proteins are both those which form part of the constituents of the skin or the hair and those which are secreted by the skin, including the scalp, and/or those which are deposited on the skin or the hair during application onto the latter of products containing proteins or protein derivatives.

Among the proteins most exposed to external attack, mention may be made in particular of collagen, glycoproteins and skin enzymes.

In order to limit the peroxidation of unsaturated lipids and proteins, it is known to apply to the skin photoprotective compositions containing at least one anti-free-radical agent or at least one screening agent.

Among the screening agents, nanopigments of metal oxides, particularly titanium dioxides, are increasingly used in skin and hair products on account of their properties of scattering and reflecting ultraviolet radiation. When used alone, they allow good protection to be obtained against ultraviolet rays. When combined with low concentrations of organic screening agents, they lead to the production of highly protective products.

However, light-irradiation of compositions containing metal oxide nanopigments catalyses the oxidation of oxidation-sensitive organic compounds, in particular:

lipids of sebaceous origin (even though an overall protective effect of the nanopigments towards lipids of sebaceous origin is observed on account of their screening activity),

lipids used in cosmetic formulations,

proteins of the skin,

proteins and protein derivatives used in cosmetic formulations.

An instability to light of compositions containing metal oxide nanopigments is also observed, manifested in particular, for titanium oxides, in the appearance of a blue-grey colour in the cosmetic products when they come into contact with daylight, this also being known as photo-blueing.

The inventors have demonstrated, according to the “head space” method, that, when exposed to ultraviolet radiation or after a few hours at 37° C., titanium oxide catalyses the production of peroxide radicals from the lipid constituents contained in the cosmetic compositions. This results—when these compositions are applied to the skin—in harmful effects such as inflammation. For a description of this method, reference may be made in particular to the articles by N'Guyen Q. L. et al. Symposium of AFECG-SFC, Bordeaux, May 1984, pp. 358-359, Evaluation of aldehydic oxidation in cosmetic products; and Warner K. et al., Journal of Food Science, 1974, V39, pp. 761-765, “Pentane formation and rancidity in vegetable oils”, the disclosures of which are specifically incorporated by reference herein.

Faced with these drawbacks, surface-treated pigments have appeared on the market, this treatment having the effect of limiting the phenomenon of light-induced activity. Thus, patent application WO 90/09777 proposes to treat titanium oxide nanopigments with phosphate anions. Patent application JP 03-183,620 describes zinc oxide nanopigments treated with a combination of alumina and silica. These surface treatments aimed at reducing the photocatalytic activity are, however, not satisfactory and the reduction of the phenomenon remains insufficient.

Thus, the compositions known to date provide insufficient protection, or even no protection at all, of the skin against the peroxidation of skin lipids and proteins and against the peroxidation of lipids and proteins contained in the cosmetic compositions intended to be applied to the skin.

The inventors have now discovered that certain polyamino polymers have antioxidant properties and allow the abovementioned problems to be solved.

The subject of the invention is the use of a polyamino polymer as an antioxidant. For the purposes of the present invention, the term polyamino polymer is understood to refer to a polymer selected from:

(A) a polyalkylenepolyamine, or one of its derivatives, selected from:

(i) polyalkylenepolyamines;

(ii) alkyl derivatives of polyalkylenepolyamines (A)(i);

(iii) addition products of alkylcarboxylic acids with polyalkylenepolyamines (A)(i);

(iv) addition products of ketones and aldehydes with polyalkylenepolyamines (A)(i);

(v) addition products of isocyanates and isothiocyanates with polyalkylenepolyamines (A)(i);

(vi) addition products of alkylene oxide or of polyalkylene oxide block polymers with polyalkylenepolyamines (A)(i);

(vii) quaternized derivatives of polyalkylenepolyamines (A)(i);

(viii) addition products of a silicone with polyalkylenepolyamines (A)(i);

(ix) copolymers of dicarboxylic acid and of polyalkylenepolyamines (A)(i);

(B) polyvinylimidazoles;

(C) polyvinylpyridines;

(D) addition products of 1-vinylimidazole monomers of formula (I):

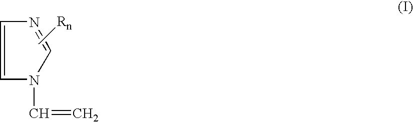

in which:

radicals R independently represent H or a linear or cyclic, saturated or unsaturated C₁-C₆ alkyl radical,

n is an integer ranging from 1 to 3, with polyalkylenepolyamines (A)(i) to (A)(ix);

(E) polymers based on amino acids with a basic side chain; and

(F) crosslinked derivatives of the polymers (A)(i) to (A)(ix), (B), (C), (D) and (E).

The polyamino polymers which can be used in the present invention can be in the linear, hyperbranched or in dendrimer form.

Hyperbranched polymers are molecular constructions having a branched structure, generally about a core. Their structure generally lacks symmetry: the monomer or base units involved in the construction of the hyperbranched polymer can be of varied nature and they are distributed non-uniformly. The branches in the polymer can be of varied nature and length. The number of base units, or monomers, can be different according to the different branching. While remaining asymmetrical, the hyperbranched polymers can have: an extremely branched structure, around a core; successive generations or layers of branching; a layer of end chains.

Hyperbranched polymers are generally obtained from the polycondensation of one or more monomers ABx, A and B being reactive groups capable of reacting together, x being an integer greater than or equal to 2, but other preparation processes can be envisaged. Hyperbranched polymers are characterized by their degree of polymerization DP=1−b, b being the percentage of non-terminal functional groups in B which have not reacted with a group A. Since the condensation is non-systematic, in contrast with the synthesis of dendrimers, the degree of polymerization is less than 100%. Usually, by the known synthetic methods, DP is between 15 and 90%. An end group T can be reacted with the hyperbranched polymer to obtain a specific functional group at the end of the chains.

Such polymers are described in particular in B. I. Voit, Acta Polymer, 46, 87-99 (1995); EP-682,059; WO-96/14346; WO-96/14345; WO-96/12754, the disclosures of which are specifically incorporated by reference herein.

Several hyperbranched polymers can be combined, by means of a covalent bond or another type of bonding, via their end groups. Such so-called “bridged” polymers come within the definition of the hyperbranched polymers according to the present invention.

Dendrimers are highly branched polymers and oligomers that are also known; they have a well-defined chemical structure and are said to be “perfect” hyperbranched polymers. As a general rule, dendrimers comprise a core, a determined number of generations of branches, or spindles, and terminal groups. The generations of spindles consist of structural units which are identical for the same generation of spindles and which can be identical or different for different generations of spindles. The generations of spindles extend radially in a geometrical progression from the core. The terminal groups of a dendrimer of the N^{th} generation are the terminal functional groups of the spindles of the N^{th} generation or terminal generation. Such polymers are described in particular in D. A. Tomalia, A. M. Naylor and W. A. Goddard III, Angewandte Chemie, Int. Ed. Engl. 29, 138-175 (1990); C. J. Hawker and J. M. J. Frechet, J. Am. Chem. Soc., 112, 7638 (1990); B. I. Voit, Acta Polymer, 46, 87-99 (1995); N. Ardoin and D. Astruc. Bull. Soc. Chim. Fr. 132, 875-909 (1995), the disclosures of which are specifically incorporated by reference herein.

Dendrimers can also, more particularly, be defined by the formula (DI) below:

C[A₁B₁(A₂B₂( . . . (Aₙ₋₁Bₙ₋₁(AₙBₙ(T)rₙ)rₙ₋₁)rₙ₋₂ . . . )r₂)r₁]s (DI)

in which:

C represents the core, linked by a number s of functional groups to s spindles A₁B₁ via the groups A₁;

s is an integer greater than or equal to 1 and less than or equal to the number of functional groups in C;

the index i (i=1, 2 . . . n) is an integer which denotes the generation of each spindle;

rᵢ (i=1, 2 . . . n−1) represents the number of functional groups in the group Bᵢ belonging to the spindle (AᵢBᵢ), ri being an integer greater than or equal to 2;

for each spindle (AᵢBᵢ) (i=1, 2 . . . n), the group Bᵢ is linked to rᵢ groups Aᵢ₊₁ of a spindle (Aᵢ₊₁Bᵢ₊₁);

each group Aᵢ (i≧2) is linked to a single group Bᵢ₋₁ of the spindle (Aᵢ₋₁Bᵢ₋₁);

the spindle of n^{th} generation AₙBₙ is linked chemically to a number rₙ of terminal groups T, rₙ being an integer greater than or equal to zero.

The dendrimer definition given above includes molecules containing symmetrical branching; it also includes molecules containing non-symmetrical branching such as, for example, dendrimers whose spindles are lysine groups, in which the branching of one generation of spindles onto the preceding generation takes place on the amines α and ε of lysine, which leads to a difference in the length of the spindles of the different branchings.

Dense star polymers, starburst polymers and rod-shaped dendrimers are included in the present definition of dendrimers. The molecules known as arborols and cascade molecules also fall within the definition of dendrimers according to the present invention.

Several dendrimers can be combined together, via a covalent bond or another type of bond, via their terminal groups in order to give species known as “bridged dendrimers” or “dendrimer aggregates”. Such species are included in the definition of dendrimers according to the present invention.

Dendrimers can be in the form of a set of molecules of the same generation, these being so-called monodispersed sets; they can also be in the form of sets of different generations, which are known as polydispersed sets. The definition of dendrimers according to the present invention includes monodispersed sets as well as polydispersed sets of dendrimers.

Reference may be made to the following documents, in which dendrimers containing amine functional groups are described, the content of these documents being incorporated by reference into the present description: U.S. Pat. No. 4,694,064; U.S. Pat. No. 4,631,337; WO-A-95/02008; WO-A-93/14147; U.S. Pat. No. 4,360,646; Proc. Natl. Acad. Sci. USA, 85, 5409-5413 (1988), the disclosures of which are specifically incorporated by reference herein.

The hyperbranched polymers and dendrimers containing amine functional groups can also consist of a core and generations of base, monomer or spindle units, of any nature, on which a terminal group T bearing an amine function has been grafted.

The polyamino polymers (A)(i) to (A)(ix), (B), (C), (D), (E) and (F) of the invention will be described in greater detail:

(A)(i)

The polyalkylenepolyamines preferably used according to the invention are polymers containing from 7 to 20,000 repeating units. Preferably, polyalkylenepolyamines comprising at least 5% of tertiary amines, advantageously at least 10% of tertiary amine functions and even more preferably at least 20%, are selected. These polymers can be linear or branched homopolymers or copolymers, or dendrimer structures.

These polymers comprise the following repeating units:

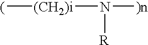

in which:

i represents an integer greater than or equal to 2, preferably i=2;

n represents an integer

R represents H or a unit

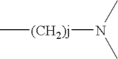

in which j represents an integer greater than or equal to 2, preferably j=2;

Among the products of the polyalkylenepolyamine family, also known as polyaziridines, mention may be made in particular of:

Polyethyleneimine, which is a hyperbranched polymer which is well known to those skilled in the art: on the subject of polyethyleneimine, reference may be made in particular to the documents: “Kirk-Othmer Encyclopedia of Chemical Technology”, 3rd Edition, Vol. 20, 1982, pp. 214-216, and “Polyethyleneimine Prospective Application”, H. N. Feigenbaum, Cosmetic & Toiletries, 108, 1993, p. 73, the disclosures of which are specifically incorporated by reference herein. Polyethyleneimine is commercially available from the company BASF under the trade names LUPASOL and POLYIMIN; polyethyleneimine is usually within an average molecular weight range from 500 to 2,000,000;

Polyethyleneimines and polypropyleneimines in the form of dendrimers are also known, these being manufactured by the company DSM. Patent applications WO 95/02008 and WO 93/14147, the disclosures of which are specifically incorporated by reference herein, describe polyalkylenepolyamines of the dendrimer family, as well as a process for their preparation.

(A)(ii) The alkyl derivatives of polyalkylene-polyamine are products that are well known to those skilled in the art. They are obtained in a known manner by alkylation, in aqueous or alcoholic medium, in the presence of an alkylating agent, preferably in the presence of NaOH, KOH or carbonate, at temperatures preferably ranging from 40° C. to 130° C. The alkylating agent can be selected, for example, from C₁-C₈ alkyl halide or alkyl sulphate derivatives such as, for example, dimethyl sulphate, diethyl sulphate, butyl bromide, hexyl bromide, 2-ethylhexyl bromide, n-octyl bromide or the corresponding chlorides. Reference may be made, for example, to DE-3,743,744 which describes the preparation of such products, the disclosure of which is specifically incorporated by reference herein.

(A)(iii) The addition products of alkylcarboxylic acids with polyalkylenepolyamines are products that are known to those skilled in the art and whose preparation is described, for example, in patent applications WO 94114873; WO 94/20681; WO 94/12560, the disclosures of which are specifically incorporated by reference herein. The addition of alkylcarboxylic acids to polyalkylenepolyamines can be carried out by reacting, in a known manner, an acid, an amide, an ester or an acid halide with the polyalkylenepolyamine polymer.

The addition products of alkylcarboxylic acids with polyalkylenepolyamines can be, for example, the addition products of saturated or unsaturated, linear or branched C₂-C₃₀ alkylcarboxylic acids with a polyethyleneimine. Among the carboxylic acids which can be used, mention may be made, for example, of acetic acid, propionic acid, butyric acid, 2-ethylhexanoic acid, benzoic acid, lauric acid, myristic acid, palmitic acid, stearic acid, oleic acid, linoleic acid, arachidonic acid and behenic acid, as well as mixtures of fatty substances such as, for example, mixtures of fatty esters available in the form of natural products, and among which mention may be made of: coconut oil, soybean oil, linseed oil, rapeseed oil, etc.

(A)(iv) The addition products of ketones and aldehydes with polyalkylenepolyamines (A)(i) can be prepared by processes known to those skilled in the art and lead to the production of α-hydroxyamine units;

(A)(v) The addition products of isocyanates and isothiocyanates with polyalkylenepolyamines (A)(i) can be prepared by processes known to those skilled in the art and lead to the production of urea and thiourea units;

(A)(vi) The addition products of alkylene oxide and of polyalkylene oxide block polymers with polyalkylenepolyamines (A)(i) can be prepared by processes known to those skilled in the art; reference may be made, for example, to the documents EP-541,018 and U.S. Pat. No. 4,144,123, the disclosures of which are specifically incorporated by reference herein, in which such molecules are described; ethoxylated polyethyleneimine derivatives are commercially available under the tradename: LUPASOL 61 (BASF);

(A)(vii) The quaternized derivatives of polyalkylenepolyamines (A)(i) can be prepared by processes known to those skilled in the art;

(A)(viii) The addition products of a silicone with polyalkylenepolyamines (A)(i) are, for example, polyethyleneimines grafted with polydimethylsiloxane units, the preparation of which is described in the document U.S. Pat. No. 5,556,616, the disclosure of which is specifically incorporated by reference herein, and sold by the company Macintyre under the trade name MACKAMER PAVS;

(A)(ix) The copolymers of dicarboxylic acid and of polyalkylenepolyamines (A)(i) can be prepared by polycondensation of dicarboxylic acids with polyalkylenepolyamines.

Among the dicarboxylic acids which can be used to prepare polyamidoamines, mention may be made of C₂ to C₁₀ dicarboxylic acids such as, for example, oxalic acid, malonic acid, itaconic acid, succinic acid, maleic acid, adipic acid, glutaric acid, sebacic acid, terephthalic acid and orthophthalic acid, as well as mixtures thereof.

The polyalkylenepolyamines used to prepare the polyamidoamines are advantageously selected from those having from 3 to 10 nitrogen atoms, such as, for example, diethylenetriamine, triethylenetetramine, dipropylenetriamine, tripropylenetetramine, dihexamethylenetriamine, aminopropylethylenediamine and bisaminopropylethylenediamine, as well as mixtures thereof. Polyethyleneimines such as those described above for the preparation of polyamidoamines can also be used.

Such compounds are described, for example, in the documents U.S. Pat. No. 4,144,423 and WO 94129422, the disclosures of which are specifically incorporated by reference herein.

(B)

The term polyvinylimidazole comprises polyvinylimidazole (PVI) homopolymers and copolymers obtained by radical polymerization of vinylimidazole monomers of the following structure:

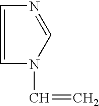

The copolymers can be, for example, vinylimidazole copolymers containing at least 5% of vinylimidazole units with monomers selected from vinylpyrrolidinone, acrylic acid and acrylamide units. The synthesis of such compounds is well known to those skilled in the art; on this subject, reference may be made in particular to the documents: J. Am. Chem. Soc., Vol. 85, 1962, p. 951; Polymer Letters Ed., Vol. 11, 1973, p. 465-469; Macromolecules, Vol. 6(2), 1973, p. 163-168; Ann. N.Y. Acad. Sci., Vol. 155, 1969, p. 431; FR-A-1,477,147; JP-69 07395; J. Macromol. Scien. Chem., Vol. A21(2), 1984, p. 253, the disclosures of which are specifically incorporated by reference herein.

(C)

The term polyvinylpyridine comprises vinylpyridine homopolymers and copolymers obtained by radical polymerization of vinylpyridine monomers (substituted in position 2 or 4 of the pyridine ring) of the following structure:

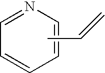

The copolymers can be, for example, vinylpyridine copolymers containing at least 5% of vinylpyridine units with monomers selected from vinylpyrrolidinone, acrylic acid and acrylamide units.

(D)

The addition products of 1-vinylimidazole monomers corresponding to formula (I):

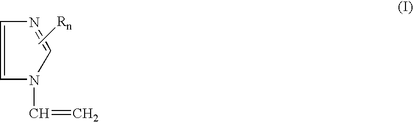

in which the radicals R independently represent H or a saturated or unsaturated, linear or cyclic, C₁-C₆ alkyl radical,

n is an integer ranging from 1 to 3, with polyalkylenepolyamines and their derivatives (A)(i) to (A)(ix).

Among the derivatives of formula (I) which can be used, mention may be made, for example, of 2-methyl-1-vinylimidazole and 2-benzyl-1-vinylimidazole.

These products are known to those skilled in the art: their preparation is described, for example, in patent application WO 94/29422, the disclosure of which is specifically incorporated by reference herein.

(E)

The polymers based on amino acids with a basic side chain are preferably selected from proteins and peptides comprising at least 5%, advantageously at least 10%, of amino acids selected from histidine, lysine and arginine.

Among these polymers, mention may be made, for example, of polylysines and polyhistadines.

(F)

Crosslinked derivatives of the polymers (A)(i) to (A)(ix), (B), (C) and (D). Among the crosslinking agents which can be used, mention may be made of halo-hydrin-, glycidyl, aziridino- and isocyanate derivatives; such crosslinking agents, as well as the methods for using them, are well known to those skilled in the art. Among the best known, mention may be made of: epichlorohydrin, α,ω-bis(chlorohydrin) polyalkylene glycol ethers, α,ω-dichloroalkanes such as, for example, 1,2-dichloroethane, 1,2-dichloropropane, 1,3-dichloropropane, 1,4-dichlorobutane and 1,6-dichlorohexane; such crosslinking agents and their use for crosslinking polyethyleneimine derivatives are described in WO 94/12560, the disclosure of which is specifically incorporated by reference herein.

Preferably, in carrying out the present invention, polyamino polymers comprising at least 5% of tertiary amines, advantageously at least 10% of tertiary amine functions and even more preferably at least 20%, are selected.

According to the invention, the polyamino polymer is advantageously selected from:

(A) (i) hyperbranched polyethyleneimines,

(ii) alkyl derivatives of polyethyleneimine;

(iii) addition products of alkylcarboxylic acids with polyethyleneimine;

(iv) addition products of ketones and aldehydes with polyethyleneimine;

(v) addition products of isocyanates and isothiocyanates with polyethyleneimine;

(vi) addition products of alkylene oxide and polyalkylene oxide block polymers with polyethyleneimine;

(vii) quaternized derivatives of polyethyleneimine;

(viii) addition products of a silicone with polyethyleneimine;

(ix) copolymers of dicarboxylic acid and of polyethyleneimine;

(B) polyvinylimidazoles.

Even more preferably, the polyamino polymer is selected from:

(A) (i) hyperbranched polyethyleneimines; and

(vi) the addition products of either ethylene oxide or of polyethylene oxide block polymers with polyethyleneimine.

Advantageously, the polyamino polymer is a polyethyleneimine.

Preferably, polyethyleneimine derivatives comprising at least 5% of tertiary amines, advantageously at least 10% of tertiary amine functions and even more preferably at least 20%, are selected.

Such polymers make it possible to inhibit oxidation reactions in general, irrespective of their origin. In particular, they have the property of inhibiting light-induced peroxidation of photo-oxidizable lipids and the light-induced peroxidation of proteins.

Thus, the subject of the invention is more particularly a polyamino polymer as described above for inhibiting the peroxidation of lipids induced by UV radiation.

Among the photo-oxidizable lipids to which the invention relates, mention may be made of unsaturated sebum lipids such as squalene, as well as the photo-oxidizable fatty substances usually used in the formulation of cosmetics.

The use of the polyamino polymers according to the invention to inhibit the light-induced peroxidation of lipids is particularly noteworthy when these lipids are in the presence of nanopigments, these nanopigments having the property of promoting the photo-oxidation reactions.

Thus, the presence of at least one polyamino polymer in a cosmetic composition comprising at least one nanopigment makes it possible, on application to the skin, to avoid the photo-peroxidation of the derivatives of skin sebum.

In addition, the presence of at least one polyamino polymer in a cosmetic composition comprising at least one photo-oxidizable fatty substance and at least one nanopigment makes it possible, both on storage and on application to the skin, to avoid the photo-peroxidation of these fatty substances.

It has also been observed that the polyamino polymers according to the invention made it possible to inhibit the photo-peroxidation of proteins, in particular the photo-peroxidation of proteins which is induced by nanopigments.

Thus, the subject of the invention is more particularly the use of a polyamino polymer as described above to inhibit the peroxidation of proteins induced by UV radiation.

Among the photo-oxidizable proteins to which the invention relates, mention may be made of skin proteins such as collagen, as well as the photo-oxidizable proteins usually used in the formulation of cosmetics.

The use of the polyamino polymers according to the invention to inhibit the light-induced peroxidation of proteins is particularly noteworthy when these proteins are in the presence of nanopigments, these nanopigments having the property of promoting the photo-oxidation reactions.

Thus, the presence of at least one polyamino polymer in a cosmetic composition comprising at least one nanopigment makes it possible, on application to the skin, to avoid the photo-peroxidation of skin collagen derivatives.

In addition, the presence of at least one polyamino polymer in a cosmetic composition comprising at least one protein or a protein derivative and at least one nanopigment makes it possible, both on storage and on application to the skin, to avoid the photo-peroxidation of these proteins.

The subject of the invention is also a cosmetic and/or dermatological composition comprising:

(i) at least one nanopigment,

(ii) at least one polyamino polymer.

The subject of the invention is also a cosmetic and/or dermatological composition comprising:

(i) at least one nanopigment,

(ii) at least one polyamino polymer,

(iii) at least one photo-oxidizable fatty substance.

The subject of the invention is also a cosmetic and/or dermatological composition comprising:

(i) at least one nanopigment,

(ii) at least one polyamino polymer,

(iii) at least one protein or a protein derivative.

According to the invention, the term “nanopigment” is understood to refer to a pigment with an average diameter preferably of less than 100 nm and more preferably ranging from 5 to 50 nm.

More particularly, the nanopigments are based on metal oxides.

The metal oxides are selected from titanium oxide, zinc oxide, cerium and zirconium oxide, or mixtures thereof.

The nanopigments can be coated or uncoated.

The coated pigments are pigments which have undergone one or more surface treatments of chemical, electronic, mechanochemical and/or mechanical nature with compounds as described, for example, in Cosmetics & Toiletries, February 1990, Vol. 105, p. 53-64, the disclosure of which is specifically incorporated by reference herein, such as amino acids, beeswax, fatty acids, fatty alcohols, anionic surfactants, lecithins, sodium, potassium, zinc, iron or aluminium salts of fatty acids, silicones, proteins (collagen, elastin), alkanolamines, silicon oxides, metal oxides or sodium hexametaphosphate, polyols and perfluoro oils.

The coated pigments are more particularly titanium oxides coated:

with silica, such as the product “SUNVEIL” from the company Ikeda,

with silica and iron oxide, such as the product “Sunveil F” from the company Ikeda,

with silica and alumina, such as the products “MICROTITANIUM DIOXIDE MT 500 SA” and “MICROTITANIUM DIOXIDE MT 100 SA” from the company Tayca, “TIOVEIL” from the company Tioxide,

with alumina, such as the products “TIPAQUE TTO-55 (B)” and “TIPAQUE TTO-55 (A)” from the company Ishihara, and “UVT 14/4” from the company Kemira,

with alumina and aluminium stearate, such as the product “UV TITAN M212” from the company Kemira,

with alumina and aluminium stearate, such as the product “MICROTITANIUM DIOXIDE MT 100 T” from the company Tayca,

with alumina and aluminium laurate, such as the product “MICROTITANIUM DIOXIDE MT 100 F” from the company Tayca,

with zinc oxide and zinc stearate, such as the product “BR 351” from the company Tayca,

with silica, alumina and silicone, such as the products “MICROTITANIUM DIOXIDE MT SAS” from the company Tayca,

with silica, alumina and perfluoropolymethyl isopropyl ether, such as the product “TiO₂ VF-25-33” from the company Toshiki,

with silica, alumina, aluminium stearate and silicone, such as the product “STT-30-DS” from the company Titan Kogyo,

with alumina and silicone, such as the products “TIPAQUE TTO-55 (S)” from the company Ishihara, and “UV TITAN M262” from the company Kemira,

with triethanolamine, such as the product “STT-65-S” from the company Titan Kogyo,

with stearic acid, such as the product “TIPAQUE TTO-55 (C)” from the company Ishihara,

with sodium hexametaphosphate, such as the product “MICROTITANIUM DIOXIDE MT 150 W” from the company Tayca.

Combinations of coated or uncoated titanium oxide nanopigments which are made water-dispersible by a hydrophilic treatment or oil-dispersible by a hydrophobic treatment, such as those described in European Patent Application 456,460, the disclosure of which is specifically incorporated by reference herein, can also be used.

Mention may also be made of metal oxide mixtures, in particular mixtures of titanium dioxide and cerium dioxide, including the weight-equivalent silica-coated mixture of titanium dioxide and cerium dioxide, sold by the company Ikeda under the name “SUNVEIL A”, as well as the mixture of titanium dioxide and zinc dioxide coated with alumina, silica and silicone, such as the product “M 261” sold by the company Kemira, or coated with alumina, silica and glycerol, such as the product “M 211” sold by the company Kemira.

The uncoated titanium oxides are sold, for example, by the company Tayca under the trade names “MICROTITANIUM DIOXIDE MT 500 B” or “MICROTITANIUM DIOXIDE MT 600 B”, by the company Degussa under the name “P 25”, by the company Wackherr under the name “TRANSPARENT TITANIUM OXIDE PW”, by the company Miyoshi Kasei under the name “UFTR” and by the company Tomen under the name “ITS”.

The uncoated zinc oxides are sold, for example, by the company Sumitomo under the name “ULTRA FINE ZINC OXIDE POWDER”, by the company Presperse under the name “FINEX 25”, by the company Ikeda under the name “MZO-25” or by the company Sunsmart under the name “Z-COTE”.

The uncoated cerium oxide is sold under the name “COLLOIDAL CERIUM OXIDE” by the company Rhône Poulenc.

According to the invention, the coated or uncoated titanium oxide nanopigments are particularly preferred.

The concentration of metal oxide nanopigments in the cosmetic compositions according to the invention preferably ranges from 0.1 to 20% by weight relative to the total weight of the composition, and more preferably from 0.25 to 15%.

The concentration of polyamino polymers in the cosmetic compositions according to the invention preferably ranges from 0.05 to 10% by weight relative to the total weight of the composition, more preferably from 0.5 to 5%.

A person skilled in the art will known how to adjust, by means of simple tests, the proportions of polyamino polymer relative to the nanopigment, and optionally relative to the other components of the composition, in particular the photo-oxidizable fatty substances and the proteins or protein derivatives. The reason for this is that the optimum proportions of the various constituents can vary, for example, depending on the molecular weight of the polymer, the amine content and/or the tertiary amine content in this polymer.

According to the invention, the expression photo-oxidizable fatty substances is understood to refer to fatty substances comprising alkyl chains containing at least one unsaturation, such as, for example, fatty acids, hydroxylated fatty acids, sterols and their derivatives, among which mention may be made of sterol esters, fatty acid esters, glycolipids, phospholipids, ceramides and terpenes.

The photo-oxidizable fatty substances to which the invention relates are those whose iodine value preferably ranges from 5 to 200. In order to measure the iodine value, reference may be made to “Iodine value: Wij's method, Handbook of Biochem. & Mol. Biol. p. 512”, the disclosure of which is specifically incorporated by reference herein. This value makes it possible to characterize the susceptibility of the fatty chains to be oxidized.

The compositions according to the invention advantageously comprise from 0.5% to 60% by weight, relative to the total weight of the composition, of at least one photo-oxidizable fatty substance.

It is thus possible to modify the iodine value of each of the compounds of the fatty phase by means of the content of this ingredient in the fatty phase. The degree of unsaturation C of the fatty phase of a composition is defined by the formula:

$$C = {\sum }_{i = 1}^{n} [ V . I . ] i * [ T . H ] i$$

in which [V.I.]i is the iodine value of the oil i and [T.H.]i is its weight percentage relative to the total weight of the fatty phase. Advantageously, the degree of unsaturation C of the fatty phase of the compositions according to the invention preferably ranges from 2.5 to 4000 and more preferably from 50 to 4000.

The invention relates mainly to the plant oils used in cosmetics, the squalene and the cholesterol present on the skin.

Among the plant oils used in cosmetics, mention may be made more particularly of apricot oil, sweet almond oil, groundnut oil, avocado oil, candlenut oil, borage oil, camellia oil, false flax oil, safflower oil, blackcurrant oil, cereal oil, chayote oil, coconut oil, rapeseed oil, coriander oil, cotton oil, cumin oil, cynara oil, evening primrose oil, perilla oil, cod liver oil, corn germ oil, jojoba oil, kiwi oil, lanolin oil, lychee oil, linseed oil, longan oil, mango oil, hazelnut oil, olive oil, palm oil, passionflower oil, grapeseed oil, cluster pine oil, Italian stone pine oil, pistachio oil, musk rose oil, sesame oil, shorea oil (floor grease), soybean oil, rice bran oil, turtle oil, sunflower oil, whale oil, tea oil, karite butter and vitamin F triglycerides.

The invention also relates to inhibition of the photo-peroxidation of proteins: this property applies to any type of protein, whether it is one of animal or plant origin, and also applies to protein hydrolysates (peptides, amino acids) and to the products derived from the condensation of a protein or a protein fragment with: a fatty acid, a silicone such as, for example, collagen fibres in sheet form sold by the company Henkel under the trade name POLYMOIST MASK A4-125; bird or fish collagen hydrolysates; hydrolysates of proteins grafted with fatty acids, such as the product sold by the company Seppic under the brand name LIPACIDE; hydrolysates of proteins grafted with a polydimethylsiloxane fragment, for example such as the product sold by the company Croda under the brand name CRODASONE.

These compositions afford effective protection against all kinds of oxidative attack without the need for other screening agents, in particular chemical screening agents. Thus, the risks of intolerance are reduced when compared with compositions containing several screening agents. In addition, with an equivalent efficacy, the compositions according to the invention are easier to prepare and less expensive than those of the prior art.

The compositions of the invention can be in various pharmaceutical forms, and in particular in the form of oil-in-water or water-in-oil emulsions, solutions, gels or vesicular dispersions. These compositions can be a care cream, a milk, a shampoo, a lotion or a serum.

According to a preferred variant, the compositions of the invention also comprise at least one complexing agent which makes it possible to complex the metals which may be present in these compositions, and in particular those in the water used in the composition, and thus make these metals inactive.

The complexing agent which can be used in the compositions of the invention is, for example, a phosphonic acid derivative and is selected in particular from 3-ethylenediaminetetra(methylenephosphonic) acid, diethylenetriaminepenta(methylenephosphonic acid) and their sodium salts. Preferably, the complexing agent is the pentasodium salt of diethylenetriaminepenta(methylenephosphonic acid).

As other complexing agents which neutralize the action of metals, diethylenetriaminepentaacetic acid, sold for example by the company Sigma, can also be used.

The complexing agents, such as ethylenediaminetetraacetic acid (EDTA) which is an iron-chelating agent, have a pro-oxidizing effect and thus cannot be used alone in the compositions of the invention for antioxidant purposes. However, it is possible to use this type of agent in combination, in particular, with a phosphonic acid derivative.

When it is present, the metal-complexing agent is at a concentration which is selected preferably from 0.005% to 0.1% by weight relative to the total weight of the composition.

The compositions of the invention can moreover contain adjuvants, used alone or as a mixture, and in particular those selected from surfactants (emulsifier or co-emulsifier) of nonionic, anionic, cationic or amphoteric type, treating agents, active agents, thickeners, suspending agents, dyes, fragrances, fillers, neutralizers, excipients (oils/water) and preserving agents.

The composition of the invention can be used as a cosmetic composition or for the manufacture of a dermatological composition for protecting the human epidermis or the hair against ultraviolet rays, as an antisun composition or as a make-up product.

The adjuvants mentioned can be incorporated at the usual doses commonly accepted, avoiding, as far as possible, those liable to release oxidation-catalysing metals. They can be lipophilic or hydrophilic.

When the cosmetic composition according to the invention is used to protect the human epidermis against UV rays or as an antisun composition, it can be in the form of a suspension or dispersion in solvents or fatty substances, in the form of a nonionic vesicular dispersion or alternatively in the form of an emulsion such as a cream or a milk or in the form of an ointment, a gel, a solid stick, an aerosol mousse or a spray.

When the cosmetic composition according to the invention is used to protect the hair, it can be in the form of a shampoo, a lotion, a gel, an emulsion, a nonionic vesicular dispersion or a lacquer for the hair and can constitute, for example, a rinse-out composition, to be applied before or after shampooing, before or after dyeing or bleaching, before, during or after permanent-waving or straightening the hair, a styling or treating lotion or gel, a blow-drying or hairsetting lotion or gel, or a permanent-waving, straightening, dyeing or bleaching composition for the hair.

When the composition is used as a make-up product for the eyelashes, the eyebrows or the skin, such as an epidermal treatment cream, a foundation, a tube of lipstick, an eyeshadow, a blusher, a mascara or an eyeliner, it can be in solid or pasty, in anhydrous or aqueous form, such as oil-in-water or water-in-oil emulsions, nonionic vesicular dispersions or alternatively suspensions.

The invention also relates to a use of these compositions as cosmetic compositions or for the manufacture of dermatological compositions intended for treatment to combat and/or prevent irritation, inflammation of the skin, acne and immunosuppression, as well as to the use of these compositions to combat and/or prevent the signs of ageing. These skin phenomena are induced in particular by solar radiation.

The invention also relates to a cosmetic treatment process for the skin to combat and/or prevent ageing, in particular light-induced ageing.

Another subject of the invention is a process for conserving compositions containing at least one light-oxidizable lipid.

On account of the fact that a cosmetic and/or dermatological composition comprising such a polyamino polymer instantaneously blocks the initiation of peroxidation reactions in the presence of radiation, such a composition is particularly useful for combating light-induced skin irritation, or even inflammation, immunosuppression and acne.

Thus, another subject of the invention is the use of a polyamino polymer as an antioxidant, in particular in a cosmetic composition or for the preparation of a dermatological composition intended for the preventive or curative treatment of irritation, inflammation, immunosuppression and/or acne, in particular irritation, inflammation, immunosuppression and/or acne induced by photo-peroxidation, more particularly the photo-peroxidation of squalene and/or collagen.

The subject of the invention is also a dermatological composition according to the invention, this composition being intended to treat the signs of ageing of the skin or the hair, in particular the signs of ageing induced by photo-peroxidation, and more particularly the signs of ageing induced by the photo-peroxidation of squalene and/or collagen.

In the particular case of the treatment of acne, a specific anti-acne agent, an antiseborrhoeic agent and/or an antibacterial agent, and in particular piroctone olamine, sold under the name OCTOPIROX by the company Hoechst, can also advantageously be incorporated into the composition of the invention.

The subject of the invention is also a cosmetic treatment process which involves combating or preventing the signs of ageing by topical application, to the skin and/or the scalp and/or the hair, of a composition comprising at least one polyamino polymer according to the invention. The subject of the invention is, in particular, a cosmetic treatment process which involves combating or preventing the signs of ageing induced by photo-peroxidation, and more particularly the signs of ageing induced by the photo-peroxidation of squalene and/or collagen, by topical application to the skin and/or the scalp and/or the hair of a composition comprising at least one polyamino polymer according to the invention.

On account of its beneficial properties, the composition of the invention is suitable for protecting any skin type and more especially greasy skin and so-called sensitive skin. It can also be used to protect the lips from cracking.

In parallel with the topical treatment, the composition of the invention can be used to conserve compositions containing at least one light-oxidizable lipid.

Thus, another subject of the invention is a process for conserving cosmetic, agrifood and/or pharmaceutical compositions containing at least one photo-oxidizable lipid or protein or protein derivative, this process involving incorporating a polyamino polymer as defined above into the said compositions.

According to the invention, the composition presents no known risks of intolerance and can be used in total safety, in particular in the cosmetic, dermatological or even veterinary fields.

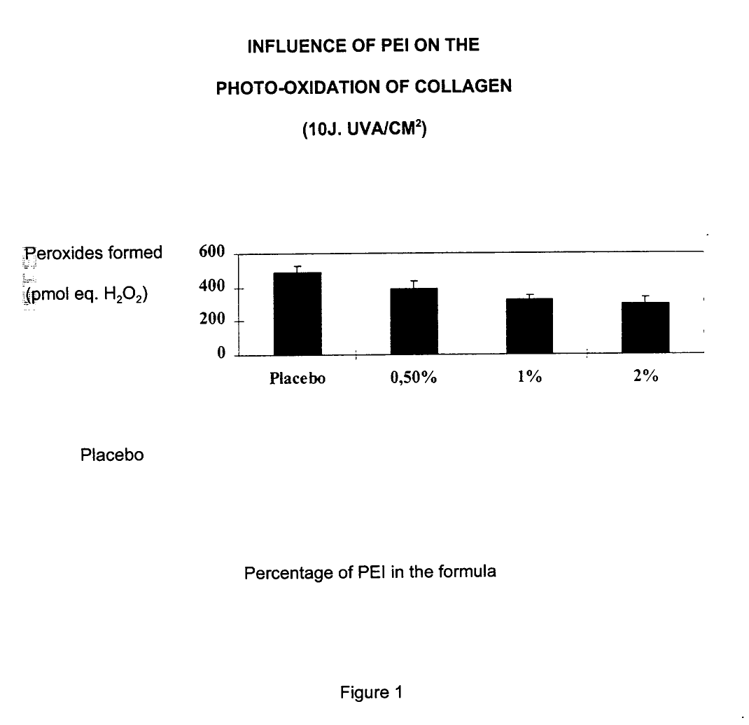

FIG. 1 illustrates the effect of polyethyleneimine on the photo-peroxidation of collagen. The percentages of polyethyleneimine (by weight relative to the total weight of the composition) in the composition subjected to the test are represented on the x-axis. The amounts of collagen peroxides (in picomole equivalents of H₂O₂) assayed in the composition are represented on the y-axis.

Other characteristics and advantages of the composition of the invention will emerge more clearly from the description, the examples and counter-examples which follow, which are given by way of non-limiting illustration. In these examples and counter-examples, the percentages are given on a weight basis.

### TESTS

In the tests described below, polyethyleneimine (PEI) of molecular weight 700, sold by the company Aldrich was used.

### Test 1

EX-VIVO Test of Inhibition of the Photo-Peroxidation of Sebum by Polyethyleneimine Under UVA

Two circular filters of 17 cm² were applied to the forehead of an individual for 10 min in order to extract a film of sebum. A thin film of placebo product (formula A) was placed on one of the filters in a proportion of about 3 mg/cm². The product containing polyethyleneimine (products B to D) was applied to the second filter. The filters were then irradiated with 5 joules UVA/cm² using a Biotronic 360 machine.

The surface lipids and the peroxides were extracted from the filters using 5 ml of HPLC-grade acetonitrile, in order to assay:

the squalene peroxides by HPLC-chemiluminescence after post-column reaction with microperoxidase-isoluminol (specific for hydroperoxides);

the residual squalene by light-scattering HPLC.

The results are expressed in terms of inhibition of the peroxidation of squalene:

$$\% inhibition = \frac{SQOOH ( formula A ) - SQOOH ( formula X )}{SQOOH ( formula A )} \times 100$$

with:

X=B,C,D;

SQOOH represents the percentage of squalene peroxides relative to the percentage of squalene (expressed in picomoles of peroxides as H₂O₂ equivalent per pg of squalene). Formulae A and X=B, C, D are formulae of the following compositions:

| CTFA name | Formula A | Formula B | Formula C | Formula D |
|---|---|---|---|---|
| Cetyl alcohol | 5% | 5% | 5% | 5% |
| Glyceryl stearate | 3% | 3% | 3% | 3% |
| PEG-50 stearate | 3% | 3% | 3% | 3% |
| Mineral oil | 20% | 20% | 20% | 20% |
| Caprylic/capric | 3% | 3% | 3% | 3% |
| triglycerides | | | | |
| Water | QSP 100 | QSP 100 | QSP 100 | QSP 100 |
| Polyethyleneimine | 0% | 0.2% | 0.5% | 1% |

The following inhibition results were obtained:

| Formula | Inhibition of peroxidation |
|---|---|
| A | 0% |
| B | 40% |
| C | 76% |
| D | 84% |

### Test 2

Inhibition, by Polyethyleneimine, of the Photo-Oxidation of Vitamin F Under UVA Induced by Light-Reactive TiO₂ Nanopigments

Test principle: compositions (A′, B′, C′) comprising vitamin F, jojoba oil and TiO₂ and optionally polyethyleneimine were spread onto a filter. Photo-oxidative degradation of the vitamin F and the jojoba oil was initiated under the joint action of the TiO₂ nanopigments and an irradiation with UVA of 100 joules per cm², using a Suntest Hereaus CPS machine. This degradation lead to the formation of pentane which was used as a marker for this oxidation. Formation of pentane was measured by chromatography using a Head-Space-40 machine (sold by the company Perkin-Elmer). The action of the PEI on the formation of pentane was thus monitored.

The compositions of the formulae were as follows:

| CTFA name | Formula A′ | Formula B′ | Formula C′ |
|---|---|---|---|
| Cetearyl alcohol | 4% | 4% | 4% |
| Ceteareth-33 | 1% | 1% | 1% |
| Glyceryl stearate | 1% | 1% | 1% |
| Cetyl alcohol | 1% | 1% | 1% |
| Jojoba oil | 6% | 6% | 6% |
| Vitamin F | 6% | 6% | 6% |
| TiO₂ P25 from Degussa | 0% | 5% | 5% |
| PEI | 0% | 0% | 2% |

| | |
|---|---|
| Water | QS 100% |

The following results were obtained:

| Formula | Pentane (in ppm) |
|---|---|
| Formula A′ | 9 |
| Formula B′ | 53 |
| Formula C′ | 10 |

Formula C′ inhibited the formation of pentane with an efficacy of about 80% relative to formula B′. It was almost equivalent to the control (formula A′) which contained no nanopigments, this corresponding to a 97% inhibition of the oxidative phenomenon which can be attributed to the action of the nanopigments.

### Test 3

Inhibition of the Photo-Peroxidation of Collagen by Polyethyleneimine Under UVA

Test Principle:

This test makes it possible to evaluate the formation of peroxide residues in collagen fibres under UVA irradiation (365 nm). The collagen (collagen sheets sold by the company Henkel under the brand name POLYMOIST MASK) was irradiated using a programmable radiometer (Biotronic UV sold by the company Vilbert Lourmat, fitted with 3 40 W/365 nm low-pressure mercury vapour lamps).

The peroxides formed were measured by chemiluminescence by means of a photon-counter camera (CCD camera sold by the company Hamamatsu):

The peroxides were decomposed by an enzyme, microperoxidase (MICROPEROXIDASE MP 11 10 H₂O sold by the company Sigma). During this reaction, oxygenated active species were produced and reacted with 6-amino-2,3-dihydro-1,4-phthalazinedione (ISOLUMINOL, sold by the company Sigma) which was also present in the reaction medium. It wats after this reaction that the isoluminol emitted photons while becoming deactivated. The photons emitted were counted by the camera; their number was proportional to the amount of peroxides initially present.

Test Formulae:

| | Formula I | | | |
|---|---|---|---|---|
| CTFA name | (placebo) | Formula 2 | Formula 3 | Formula 4 |
| Cetyl alcohol | 5% | 5% | 5% | 5% |
| Glyceryl stearate | 3% | 3% | 3% | 3% |
| PEG-50 stearate | 3% | 3% | 3% | 3% |
| Mineral oil | 20% | 20% | 20% | 20% |
| Caprylic/capric | 3% | 3% | 3% | 3% |
| triglycerides | | | | |
| Water | QSP 100 | QSP 100 | QSP 100 | QSP 100 |
| Polyethyleneimine | 0% | 0.5% | 1% | 2% |

Method of Operation:

Preparation of the chemiluminescence reaction mixture:

34.8 mg of Isoluminol are weighed out and were dissolved, with magnetic stirring, in about 50 ml of 100 mM borate buffer. 2 mg of Microperoxidase were weighed out and were dissolved in about 5 ml of 100 mM borate buffer. The two reagents were then mixed together in a 200 ml volumetric flask whose volume was adjusted with borate buffer. The 200 ml are placed in a 500 ml flask. 200 ml of borate buffer are then added thereto again. The reaction mixture, whose final volume was now 400 ml, was homogenized by magnetic stirring and then placed in darkness for at least 24 hours before use.

Discs 6 mm in diameter were cut out of a sheet of collagen using a punch. These discs were then placed in glass Petri dishes, either in aqueous solution or on a 1% agarose gel. Formulae 1 to 4 were applied on each sample of collagen. For each formula tested, 6 discs were used per dish. Each test formula was applied using a fingerstall so as to saturate the collagen disc. The Petri dishes were then exposed to UVA radiation (365 nm) of intensity: 10 joules/cm². After the irradiation, the collagen discs were placed in a 96-well plate, in a lightproof box, under a photon-counter camera.

150 microlitres of the chemiluminescence reaction mixture were then distributed into each of the wells involved in the measurement. At the same time, the count of the photon camera was triggered. Counting of the photons was carried out over a period of 4 minutes.

The count values obtained were then converted into picomole equivalents of hydroperoxide (H₂O₂) by linear regression carried out using a standard range.

The results are illustrated in FIG. 1. It is observed that polyethyleneimine inhibited the formation of collagen peroxides.

We claim:

1. A cosmetic and/or dermatological composition comprising, in a cosmetically and/or dermatologically acceptable support:
at least one nanopigment in said composition,
at least one polyamino polymer in said composition selected from:
(A) polyalkylenepolyamine polymers selected from:
(i) polyalkylenepolyamines;
(ii) alkyl derivatives of polyalkylenepolyamines;
(iii) addition products of alkylcarboxylic acids with polyalkylenepolyamines;
(iv) addition products of ketones and aldehydes with polyalkylenepolyamines;
(v) addition products of isocyanates and isothiocyanates with polyalkylenepolyamines;
(vi) addition products of alkylene oxide and polyalkylene oxide block polymers with polyalkylenepolyamines;
(vii) quaternized derivatives of polyalkylenepolyamines;
(viii) addition products of a silicone with polyalkylenepolyamines;
(ix) copolymers of dicarboxylic acid and of polyalkylenepolyamines;
(B) polyvinylimidazoles;
(C) polyvinylpyridines;
(D) addition products of 1-vinylimidazole monomers of formula (I): 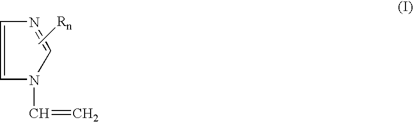
in which:
radicals R independently represent H or a linear or cyclic, saturated or unsaturated C₁-C₆ alkyl radical,
n is an integer ranging from 1 to 3, with polyalkylenepolyamines (A)(i) to (A)(ix);
(E) amino acid polymers with a basic side chain; and
(F) crosslinked derivatives of polymers (A)(i) to (A)(ix), (B), (C), (D) and (E).

2. A cosmetic and/or dermatological composition according to claim 1, wherein said polyalkylenepolyamines comprise from 7 to 20,000 repeating units

3. A cosmetic and/or dermatological composition according to claim 1, wherein said at least one polyamino polymer is selected from polyamino polymers comprising at least 5% of tertiary amine functions.

4. A cosmetic and/or dermatological composition according to claim 3, wherein said at least one polyamino polymer is selected from polyamino polymers comprising at least 10% of tertiary amine functions.

5. A cosmetic and/or dermatological composition according to claim 4, wherein said at least one polyamino polymer is selected from polyamino polymers comprising at least 20% of tertiary amine functions.

6. A cosmetic and/or dermatological composition according to claim 1, wherein said at least one polyamino polymer is selected from:
(A) (i) hyperbranched polyethyleneimines;
(ii) quaternized polyethyleneimine derivatives;
(iii) addition products of alkylcarboxylic acids with polyethyleneimine;
(iv) addition products of ketones and aldehydes with polyethyleneimine;
(v) addition products of isocyanates and isothiocyanates with polyethyleneimine;
(vi) addition products of alkylene oxide and polyalkylene oxide block polymers with polyethyleneimine;
(vii) quaternized polyethyleneimine derivatives;
(viii) addition products of a silicone with polyethyleneimine;
(ix) copolymers of a dicarboxylic acid and polyethyleneimine; and
(B) polyvinylimidazoles.

7. A cosmetic and/or dermatological composition according to claim 6, wherein said at least one polyamino polymer is selected from:
(A) (i) hyperbranched polyethyleneimines; and
(A) (vi) addition products of either ethylene oxide or polyethylene oxide block polymers with polyethyleneimine.

8. A cosmetic and/or dermatological composition according to claim 7, wherein said at least one polyamino polymer is selected from hyperbranched polyethyleneimines.

9. A cosmetic and/or dermatological composition according to claim 1, wherein said at least one nanopigment is selected from metal oxides.

10. A cosmetic and/or dermatological composition according to claim 9, wherein said at least one nanopigment is selected from titanium oxide, zinc oxide, cerium oxide and zirconium oxide.

11. A cosmetic and/or dermatological composition according to claim 10, wherein said at least one nanopigment is titanium oxide.

12. A cosmetic and/or dermatological composition according to claim 1, wherein said at least one nanopigment is present in an amount ranging from 0.1 to 20% by weight relative to the total weight of said cosmetic and/or dermatological composition.

13. A cosmetic and/or dermatological composition according to claim 12, wherein said at least one nanopigment is present in an amount ranging from 0.25 to 15% by weight relative to the total weight of said cosmetic and/or dermatological composition.

14. A cosmetic and/or dermatological composition according to claim 1, wherein said at least one polyamino polymer is present in an amount ranging from 0.05 to 10% by weight relative to the total weight of said cosmetic and/or dermatological composition.

15. A cosmetic and/or dermatological composition according to claim 14, wherein said at least one polyamino polymer is present in an amount ranging from 0.5 to 5% by weight relative to the total weight of said cosmetic and/or dermatological composition.

16. A cosmetic and/or dermatological composition according to claim 1, wherein said cosmetic and/or dermatological composition further comprises at least one photo-oxidizable fatty substance.

17. A cosmetic and/or dermatological composition according to claim 16, wherein said at least one photo-oxidizable fatty substance is selected from photo-oxidizable fatty substances with iodine values ranging from 5 to 200.

18. A cosmetic and/or dermatological composition according to claim 1, containing a fatty phase including at least one oil and wherein the degree of unsaturation (C) of the fatty phase of said cosmetic and/or dermatological composition, defined by the formula:
$$C = {\sum }_{i = 1}^{n} [ V . I . ] i * [ T . H ] i$$
in which
[V.I.]i represents the iodine value of the oil i, and
[T.H.]i represents the weight percentage relative to the total weight of said fatty phase, ranges from 2.5 to 4000.

19. A cosmetic and/or dermatological composition according to claim 18, wherein said degree of unsaturation (C) of the fatty phase of said composition ranges from 50 to 4000.

20. A cosmetic and/or dermatological composition according to claim 16, wherein said at least one photo-oxidizable fatty substance is present in an amount ranging from 0.5 to 60% by weight relative to the total weight of said cosmetic and/or dermatological composition.

21. A cosmetic and/or dermatological composition according to claim 1, wherein said cosmetic and/or dermatological composition further comprises at least one protein or protein derivative.

22. A cosmetic and/or dermatological composition according to claim 1, wherein said composition further comprises at least one oil.

23. A cosmetic and/or dermatological composition according to claim 22, wherein said at least one oil is selected from apricot oil, sweet almond oil, groundnut oil, avocado oil, candlenut oil, borage oil, camellia oil, false flax oil, safflower oil, blackcurrant oil, cereal oil, chayote oil, coconut oil, rapeseed oil, coriander oil, cotton oil, cumin oil, cynara oil, evening primrose oil, perilla oil, cod liver oil, corn germ oil, jojoba oil, kiwi oil, lanolin oil, lychee oil, linseed oil, longan oil, mango oil, hazelnut oil, olive oil, palm oil, passionflower oil, grapeseed oil, cluster pine oil, Italian stone pine oil, pistachio oil, musk rose oil, sesame oil, shorea oil (floor grease), soybean oil, rice bran oil, turtle oil, sunflower oil, whale oil, tea oil, karite butter and vitamin F triglycerides.

24. A cosmetic and/or dermatological composition according to claim 1, wherein said composition further comprises at least one metal-complexing agent.

25. A cosmetic and/or dermatological composition according to claim 21, wherein said at least one metal-complexing agent is selected from 3-ethylenediaminetetra(methylenephosphonic acid), diethylenetriaminepenta(methylenephosphonic acid) and diethylenetriaminepentaacetic acid, and sodium salts thereof.

26. A cosmetic and/or dermatological composition according to claim 25, wherein said at least one metal-complexing agent is present in an amount ranging from 0.005% to 0.1% by weight relative to the total weight of said cosmetic and/or dermatological composition.

27. A cosmetic and/or dermatological composition according to claim 1, wherein said cosmetic and/or dermatological composition is in the form of an oil-in-water or water-in-oil emulsion, a solution, a gel, a vesicular dispersion, a solid, a foam, a mousse, or a spray.

28. A cosmetic and/or dermatological composition according to claim 1, wherein said cosmetic and/or dermatological composition further comprises at least one conventional cosmetic adjuvant.

29. A method of treating the signs of ageing of the skin or the hair comprising applying at least one dermatological composition according to claim 1 to said skin or hair.

30. A method according to claim 29, wherein said signs of ageing are induced by photo-peroxidation.

31. A method according to claim 30, wherein said signs of ageing are induced by photo-peroxidation of squalene or collagen.

32. A topical cosmetic treatment process for combatting or preventing signs of ageing comprising:
topically applying at least one cosmetic composition according to claim 1 to the skin, scalp, or hair.

33. A cosmetic or dermatological composition for protecting the human epidermis or the hair against ultraviolet rays comprising at least one nanopigment in said composition and at least one polyamino polymer in said composition as defined in claim 1.

34. A cosmetic or dermatolgocial composition according to claim 33 wherein said composition is an antisun composition or a make-up product.

35. A cosmetic or dermatolgocial composition for combatting or preventing light-induced irritation, inflammation, immunosuppression or acne comprising comprising at least one nanopigment in said composition and at least one polyamino polymer in said composition as defined in claim 1.

36. A process for conserving cosmetic, pharmaceutical, veterinary or agrifood compositions containing at least one photo-oxidizable lipid, protein or protein derivative comprising incorporating in said composition at least one polyamino polymer selected from:
(A) polyalkylenepolyamine polymers selected from:
(i) polyalkylenepolyamines;
(ii) alkyl derivatives of polyalkylenepolyamines;
radicals R independently represent H or a linear or cyclic, saturated or unsaturated C₁-C₆ alkyl radical,
n is an integer ranging from 1 to 3, with polyalkylenepolyamines (A)(i) to (A)(ix);
(E) amino acid polymers with a basic side chain; and
(F) crosslinked derivatives of polymers (A)(i) to (A)(ix), (B), (C), (D) and (E).

37. An antioxidant composition comprising at least one polyamino polymer in said composition selected from:
(A) polyalkylenepolyamine polymers selected from:
(i) polyalkylenepolyamines;
(ii) alkyl derivatives of polyalkylenepolyamines;
(iii) addition products of alkylcarboxylic acids with polyalkylenepolyamines;
(iv) addition products of ketones and aldehydes with polyalkylenepolyamines;
(v) addition products of isocyanates and isothiocyanates with polyalkylenepolyamines;
(vi) addition products of alkylene oxide and polyalkylene oxide block polymers with polyalkylenepolyamines;
(vii) quaternized derivatives of polyalkylenepolyamines;
(viii) addition products of a silicone with polyalkylenepolyamines;
(ix) copolymers of dicarboxylic acid and polyalkylenepolyamines;
(B) polyvinylimidazoles;
(C) polyvinylpyridines;
(D) addition products of 1-vinylimidazole monomers of formula (I): 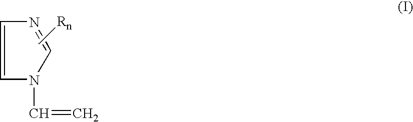
in which the radicals R independently represent H or a linear or cyclic, saturated or unsaturated C₁-C₆ alkyl radical,
n is an integer ranging from 1 to 3, with polyalkylenepolyamines (A)(i) to (A)(ix);
(E) amino acid polymers with a basic side chain; and
(F) crosslinked derivatives of the polymers (A)(i) to (A)(ix), (B), (C), (D) and (E).

38. A method for providing antioxidation properties to a substrate or composition subject to oxidation comprising the step of contacting said substrate or composition with at least one polyamino polymer selected from:
(A) polyalkylenepolyamine polymers selected from:
(i) polyalkylenepolyamines;
(ii) alkyl derivatives of polyalkylenepolyamines;
(iii) addition products of alkylcarboxylic acids with polyalkylenepolyamines;
(iv) addition products of ketones and aldehydes with polyalkylenepolyamines;
(v) addition products of isocyanates and isothiocyanates with polyalkylenepolyamines;
(vi) addition products of alkylene oxide and polyalkylene oxide block polymers with polyalkylenepolyamines;
(vii) quaternized derivatives of polyalkylenepolyamines;
(viii) addition products of a silicone with polyalkylenepolyamines;
(ix) copolymers of dicarboxylic acid and polyalkylenepolyamines;
(B) polyvinylimidazoles;
(C) polyvinylpyridines;
(D) addition products of 1-vinylimidazole monomers of formula (I): 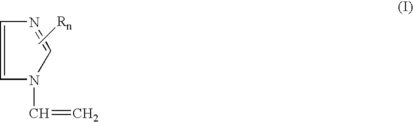
in which the radicals R independently represent H or a linear or cyclic, saturated or unsaturated C₁-C₆ alkyl radical,
n is an integer ranging from 1 to 3, with polyalkylenepolyamines (A)(i) to (A)(ix);
(E) amino acid polymers with a basic side chain; and
(F) crosslinked derivatives of the polymers (A)(i) to (A)(ix), (B), (C), (D) and (E).

39. A method according to claim 38, wherein said substrate is human epidermis or hair.

40. A method for curative or preventive treatment of irritation, inflammation, immunosuppression or acne comprising applying at least one cosmetic and/or dermatological composition according to claim 1 to a substrate or composition in need of such treatment.

41. A method according to claim 40, wherein said substrate is human epidermis or hair.

42. A method for inhibiting the peroxidation of lipids induced by UV radiation comprising the step of contacting said lipids with at least one cosmetic and/or dermatological composition according to claim 1.

43. A method for inhibiting the peroxidation of proteins and protein derivatives induced by UV radiation comprising applying to said proteins and protein derivatives at least one cosmetic and/or dermatological composition according to claim 1.

44. A process for conserving a cosmetic, an agrifood and/or a pharmaceutical composition comprising at least one photo-oxidizable lipid or protein or protein derivative, comprising including in said composition at least one polyamino polymer according to claim 1.
